(19) United States
(12) Patent Application Publication
(10) Pub. No.: US 2002/0146697 A1
(43) Pub. Date: Oct. 10, 2002

(54) Method for identification and detection of microorganisms using gyrase gene as an indicator

(76) Inventors: Satoshi Yamamoto, Iwate (JP); Hiroaki Kasai, Iwate (JP); Shoko Nakamura, Iwate (JP); Makoto Suzuki, Iwate (JP); Tohru Hamada, Iwate (JP)

Correspondence Address: CHRIS T. MIZUMOTO, Fish & Richardson P.C., Suite 2800, 45 Rockefeller Plaza, New York, NY, 10111, US

(21) Appl. No.: 09/823,829

(22) Filed: Mar. 30, 2001

Related U.S. Application Data
(63) Continuation-in-part of application No. 09/208,688, filed on Dec. 10, 1998, now abandoned.

(30) Foreign Application Priority Data
Dec. 12, 1997 (JP) 343316/1997

(51) Int. Cl. C12Q 1/68; C12P 19/34

(52) U.S. Cl. 435/6; 435/91.2

(57) ABSTRACT

With the nucleotide sequence of gyr B, it is possible to classify or identify an unidentified microorganism strain quickly and accurately. Furthermore, PCR primers for monitoring a specific microorganism, which are needed in risk assessment in various bioprocesses, can be designed easily. Also, changes in mycelial tufts can be accurately monitored.

### BACKGROUND OF THE INVENTION

1. Field of the Invention

The present invention is involved in a method for the identification and detection of organisms using the sequences of their genes encoding the B subunit of the DNA gyrase.

This invention is useful in medical fields as well as various industrial fields where the identification/classification or detection/monitoring of specific microorganisms (bacteria, yeasts, fungi, archaea and bacteria), especially bacteria, is necessary.

2. Prior Art

Conventionally, the identification/classification of living organisms has been carried out using the combination of biochemical and morphological tests. However, these tests often did not provide unequivocal answers to the taxonomic positions of tested organisms.

Recently, the taxonomy of organisms, in particular of bacteria, using rRNA sequences became fashionable. There are many reasons why rRNA molecules have been selected as standard molecules for the molecular taxonomy. They are constituents of all organisms. They exist in abundance, and therefore, can readily be isolated and characterized. For sequence comparison, many conserved regions of rRNA molecules allowed the alignment between distantly related organisms, while variable regions are useful for the distinction of closely related organisms (van de Peer, Y., S. Chapelles, and R. de Wacher. 1996. A quantitative map of nucleotide substitution rates in bacterial rRNA. Nucleic Acids Res. 24: 3381-3391; and Gutell, R. R., N. Larsen, and C. R. Woese. 1994. Lessons from an evolving rRNA: 16S and 23S rRNA structures from a comparative perspective. Microbiol. Rev. 58: 10-26). Furthermore, there is a few evidence for the horizontal transfer of rRNA genes although many other genes are expected to have frequently been transferred from one species to other distantly related species. At present, rRNA sequences are accumulating rapidly and they are accessible via an international database (Ribosomal Database project, http://rdp.life.uiuc.edu/).

However, as is clear from the fact that the evolution speed of rRNA genes is extremely slow, there is little difference in the rRNA sequences between closely related organisms. Therefore, in many times, species belonging to the same genus could not be discriminated by the analysis using rRNA sequences. For example, it is said that bacteria sharing more than 97% of identity in their 16S rRNA sequences (bacterial small subunit rRNA) might belong to the same species. However, there are cases of bacteria exhibiting more than 99% identity in their 16S rRNA sequences, and yet belonging to two distinct species as revealed from DNA hybridization analysis. Evidently, due to the slow speed of divergent evolution of the 16S rRNA gene, the resolution of 16S rRNA-based analysis between closely related organisms is lower than that of DNA hybridization analysis (Stackebrandt, E. and Goebel, B. M. 1994. Taxonomic note: a place far DNA-DNA reassociation and 16S rRNA sequence analysis in the present species difinition in bacteriology. Int. J. Syst. Bacteriol. 37: 463-464).

Other problems exist in the rRNA-based phylogenetic analysis. To establish a phylogenetic relationship based on rRNA sequences, these sequences should be aligned. The alignment of rRNA sequences composed from four different constituents (AUCG), however, is not easy, and requires some expertise. The correct sequencing of rRNA genes is also difficult largely due to their highly ordered structure. Furthermore, polymorphism of rRNA was found in some organisms.

In contrast, protein-encoding genes have evolved more rapidly than rRNA-encoding genes, since they allow the so-called neutral mutations that do not cause any amino acid substitutions in their gene products. It is then expected that, by using such protein-encoding genes, more precise phylogenetic analysis can be performed than by using rRNA sequences. Thus, the present inventors have developed and applied a method for the identification/classification or detection/monitoring of organisms using the sequences of gyr B genes encoding the B subunit of DNA gyrases (Yamamoto, S. and Harayama, S. 1995. PCR Amplification and Direct Sequencing of gyr B Genes with Universal Primers and Their Application to the Detection and Taxonomic Analysis of Pseudomonas putida Strains. Appl. Environ. Microbiol. 61: 1104-1109; Yamamoto, S. and Harayama, S. 1996. Phylogenetic Analysis of Acinetobacter Strains Based on the Nucleotide Sequences of gyr B Genes and on the Amino acid Sequences of Their Products. Int. J. Syst Bacteriol. 46: 506-511; Yamamoto, S. and Harayama S. 1998. Phylogenetic relationships of Pseudomonas putida strains deduced from the nucleotide sequences of gyr B, rpoD and 16S rRNA genes. Int. J. Syst Bacteriol. 48: 813-819; Yamamoto S., Bouvet P. J. M. & Harayama, S. 1998. Phylogenetic structures of the genus Acinetobacter based on the gyr B sequences: Comparison with the grouping by DNA-DNA hybridization. Int. J. Syst. Bacteriol. (in press); Harayama, S. and Yamamoto, S. 1996. P hylogenetic Identification of Pseudomonas Strains Based on a Comparison of gyr B and rpoD Sequences. p. 250-258 in Molecular Biology of Pseudomonads, edited by T, Nakazawa, K. Furukawa, D Haas, S. Silver. ASM Press, Washington, D.C.: and Watanabe, K., Yamamoto, S., Hino, S. and Harayama, S. 1998. Population dynamics of phenol-degrading bacteria in activated sludge determined by gyr B-targeted quantitative PCR. Appl. Environ. Microbiol. 64: 1203-1209).

DNA topoisomerases are essential for the replication, transcription, recombination and repair of DNA and control the level of supercoiling of DNA molecules by cleaving and resealing the phosphodiester bond of DNA. They are classified into type I (EC 5.99. 1.2) and type II (EC 5.99.1.3) according to their enzymatic properties. The DNA gyrase is a type II topoisomerase that is capable of introducing negative supercoiling into a relaxed closed circular DNA molecule. This reaction is coupled with ATP hydrolysis. DNA gyrase can also relax supercoiled DNA without ATP hydrolysis. DNA gyrase consists of two subunit proteins in the quaternary structure of A2B2. The A subunit (GyrA) has a molecular weight of approximately 100 kDa while the B subunit (GyrB) has a molecular weight of either 90 kDa or 70 kDa (Wigley, D. B. 1995. Structure and mechanism of DNA topoisomerases. Ann. Rev. Biomol. Struct. 24: 185-208). The genes for DNA gyrase or its isofunctional enzymes should exist in all organisms as they are indispensable for the cell proliferation.

As described above, the present Inventors have already developed and applied successfully the method for the identification/classification or detection/monitoring of organisms using gyr B sequences. In this method, a gyr B gene fragment of an organism of interest is amplified by PCR using primers designed from the two amino acid sequences, His-Ala-Gly-Gly-Lys-Phe-Asp and Met-Thr-Asp-Ala-Asp-Val-Asp-Gly, which are highly conserved among the GyrB sequences of many organisms. Subsequently, the amplified fragments are subjected to direct sequencing. Since the gyr B genes code for proteins, they have frequently undergone neutral mutations. Thus, the nucleotide sequence of the gyr B genes vary considerable even among related organisms. For this reason, the above method has been shown to be effective for discriminating organisms at a level of species of subspecies.

The above-mentioned PCR primers designed from the highly conserved amino acid sequences of GyrB were effective in many but not all bacterial species for the PCR amplification of gyr B. From DNA of some bacterial species, no PCR amplification was observed using these primers.

Besides, there was another problem associated with these primers. The genes for type IV topoisomerese (ParE) were also amplified from DNA of some bacterial species by using these primers. Topoisomerase IV (ParE) is a bacterial enzyme that appears to be closely related to DNA gyrase. This enzyme involves in the partition of chromosomes into daughter cells. If a ParE gene but not gyr B gene is amplified from a DNA, and if a phylogenetic analysis is carried out without recognizing that the amplified sequence is parE but not gyr B, it will bring some confusion to the phylogenetic analysis. To avoid such problem associated with the amplification of paralogous genes, primers which do not amplify ParE should be developed.

### OBJECTIVE AND SUMMARY OF THE INVENTION

It is an object of the present invention to solve the above-described problems of the primers and to provide a means which enables the identification/classification and detection/monitoring of a wide range of organisms using gyr B sequences.

Comparing the amino acid sequence data of GyrB collected by the inventors with those of ParE, the inventors have found that a gyrB gene DNA fragment only can be isolated from a wide range of microorganisms by performing PCR using two specific primers (forward and reverse primers), thereby completing the present invention.

First, the present invention relates to a method for the indentification and detection of a microorganism, comprising the following steps (1) to (5):

(1) synthesizing two specific primers,

(2) amplifying gyrB gene DNA from the microorganism using the above two primers to produce a gyrB gene DNA fragment,

(3) isolating the above DNA fragment,

(4) determining the nucleotide sequence of the above DNA fragment, and

(5) identifying and detecting the microorganism by comparing the nucleotide sequence of the amplified gyrB gene DNA fragment to known gyrB gene DNA fragment sequences.

Second, the present invention relates to a method for the identification and detection of a microorganism, comprising the following steps (1) to (7):

(1) synthesizing two specific primers,

(2) amplifying gyrB gene DNA from the microorganism using the above two primers to produce a gyrB gene DNA fragment,

(3) synthesizing two pairs of specific primers,

(4) amplifying the gyrB gene DNA fragment produced in step (2) using the above two pairs of primers to produce two gyrB gene DNA fragments,

(5) isolating the above two DNA fragments,

(6) determining the nucleotide sequences of the above two DNA fragments, and

(7) identifying and detecting the microorganism by comparing the nucleotide sequences of the above two gyrB gene DNA fragments to known gyrB gene DNA fragment sequences.

Third, the present invention relates to a method for the indentification and detection of a microorganism, comprising the following steps (1) to (5):

(1) synthesizing two pairs of specific primers,

(2) amplifying gyrB gene DNA from the microorganism using the above two pairs of primers to produce gyrB gene DNA fragments,

(3) isolating the above two DNA fragments,

(4) determining the nucleotide sequences of the above two DNA fragments, and

(5) identifying the microorganism by comparing the nucleotide sequences of the above two gyrB gene DNA fragments to known gyrB gene DNA fragment sequences.

### BRIEF DESCRIPTION OF THE DRAWINGS

FIG. 1 shows the locational relationship between the amino acid sequence (a) through (l) and the amino acid sequences of GyrB of several organisms.

### DETAILED DESCRIPTION OF THE INVENTION

Hereinbelow, the present invention will be described in detail.

The method for identification and detection of a microorganism of the present invention is a method for identifying and detecting of a microorganism based on the nucleotide sequence of a gyrB gene. The nucleotide sequence data of a gyrB gene may be obtained from a DNA fragment amplified by PCR (one fragment method), or may be obtained from two fragments having a mutually overlapping portion (two fragments method).

In order to identify and detect a microorganism with high accuracy, a nucleotide sequence of approx. 1 kb DNA fragment is required. In 1994, Yamamoto and Harayama filed a patent application for an invention relating to primers to amplify a gyrB fragment, which is a gene encoding the B subunit of DNA gyrases (Japanese Patent Application Laid-Open (Kokai) No. 1995-213299). However, the subsequent study has clarified that, according to the disclosure of the above application alone, the identification and detection of a microorganism may be difficult for the following reasons:

(1) It is difficult to determine a nucleotide sequence by a one-time sequencing reaction.

(2) Some strains are inefficiently amplified with only the known primer sequences.

(3) In some cases, it is difficult to determine a nucleotide sequence, since parE, being a homologous gene of gyrB, is amplified together with gyrB. In other cases, an incorrect identification of a microorganism is performed, since parE is selectively amplified.

To overcome the above-stated problems, in addition to the primer sequences disclosed by Yamamoto and Harayama in 1995, the present invention provides: primer sequences conserved among gyrB genes of a large number of bacteria; primer sequences specific for gyrB genes, not appearing in parE genes, or primer sequences appearing in both gyrB and parE genes, which allow easy distinction of an amplified parE fragment from an amplified gyrB fragment; and a method for specific amplification of a gyrB gene by the combined use of the above primer sequences.

To overcome the above-stated problems, sometimes it may be more preferable to amplify separately two DNA fragments having a mutually overlapping portion, and to determine the nucleotide sequence of each fragment, followed by connecting the obtained two sequences before using the data.

In order to amplify the two fragments, DNA obtained from the microorganism (which also contains various DNAs as well as gyrB gene DNA) may be used as is, as a template (“one step—two fragments method”), but as another option, after performing PCR using primers capable of specifically amplifying a gyrB gene DNA, the obtained amplified products may be used as a template (“two steps—two fragments method”).

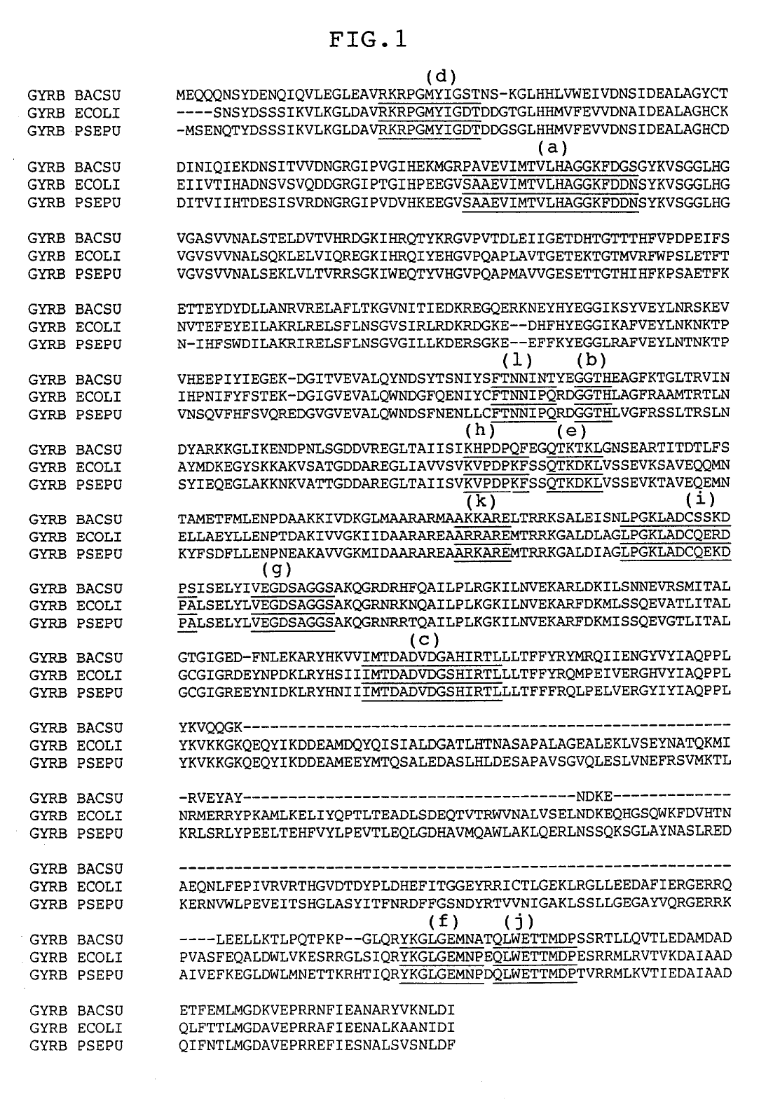

Primers used for the above three types of DNA amplification methods (“one fragment method”, “one step—two fragments method” and “two steps—two fragments method”) are shown in FIG. 1. The FIGURE shows the amino acid sequence of each GyrB of 3 kinds of strains, Bacillus subtilis 168, Escherichia coli K-12 and Pseudomonas putida PRS2000, and (a)-(l) in the FIGURE respectively correspond to amino acid sequences (a)-(l) shown as follows:

(a) (Pro or Ser)-(Ala or Thr)-(Ala, Val or Leu)-Glu or Asp)-(Val or Thr)-(Ile or Val)-(Met, Leu or Phe)-Thr-(Val, Gln or Ile)-Leu-His-Ala-Gly-Gly-Lys-Phe-(Asp or Gly)-(Asp, Gly, Asn or Ser)-(Ser, Lys, Gly, Asp or Asn) [SEQ ID NO.69]

(b) Gly-Gly-Thr-His [SEQ ID NO.70]

(c) (Ile or Leu)-Met-Thr-Asp-Ala-Asp-Val-Asp-Gly-(Ala or Ser)-His-Ile-Arg-Thr-Leu [SEQ ID NO.71]

(d) Arg-Lys-Arg-Pro-(Gly or Ala)-Met-Tyr-Ile-Gly-(Ser or Asp)-Thr [SEQ ID NO.72]

(e) Gln-(Thr or Pro)-(Lys or Asn)-(Thr, Asp, Gly, Lys, Ser, Phe or Tyr)-Lys-Leu [SEQ ID NO.73]

(f) (Tyr or Phe)-Lys-Gly-Leu-Gly-Glu-Met-Asn-(Ala or Pro) [SEQ ID NO.74]

(g) Val-Glu-Gly-Asp-Ser-Ala-Gly-Gly-Ser [SEQ ID NO.75]

(h) Lys-(His or Val)-Pro-Asp-Pro-(Gln or Lys)-Phe [SEQ ID NO.76]

(i) Leu-Pro-Gly-Lys-Leu-Ala-Asp-Cys-(Ser or Gln)-(Ser or Glu)-(Lys or Arg)-Asp-Pro-(Ala or Ser) [SEQ ID NO.77]

(j) Gln-Leu-(Trp or Arg)-(Glu or Asp)-Thr-Thr-(Met or Leu)-(Asp or Asn)-Pro [SEQ ID NO.78]

(k) Ala-(Lys or Arg)-(Lys or Arg)-Ala-Arg-Glu [SEQ ID NO.79]

(l) Phe-Thr-Asn-Asn-Ile-(Pro or Asn)-(Thr or Gln) [SEQ ID NO.80].

Two primers used for the “one fragment method” include the ones which are synthesized based on a single pair of amino acid sequences selected from the group consisting of sequence pairs (a) and (f), (a) and (j), (d) and (c), (d) and (f), and (d) and (j). The gyrB gene of members of proteobacteria has an approx. 500 bp insertion sequence located between a sequence pair (c) and (f), and another sequence pair (c) and (j), on the other hand, parE which is a homologous gene thereof does not have such sequence (Kato, J.-i., Nishimura, Y., Imamura, R., Niki, H., Hiraga, S., and Suzuki, H. New topoisomerase essential for chromosome segregation in E. coli Cell 63, 393-404 (1990)). Accordingly, in a case where two primers synthesized based on amino acid sequence pairs (a) and (f), (a) and (j), (d) and (f), or (d) and (j) are used for identification and detection of a microorganism belonging to proteobacteria, DNA fragments of gyrB genes can easily be separated from those of parE genes by electrophoresis and so on, since the length of the amplified DNA fragments of the two types of genes is different.

Two primers used for the “one step—two fragments method” include the following amino acid sequence combinations:

| | Amplified fragment 1 | Amplified fragment 2 |
|---|---|---|
| Combination 1 | sequences (d) and (e) | sequences (h) and (c), sequences (h) |
| | | and (f), or sequences (h) and (g) |
| Combination 2 | sequences (a) and (i) | sequences (k) and (c), sequences (k) |
| | | and (f), or sequences (k) and (g) |
| Combination 3 | sequences (a) and (i) | sequences (l) and (c), sequences (l) |
| | | and (f), or sequences (l)and (g) |

Among amino acid sequences in the above table, sequences (i), (e), (h), (k) and (l) do not exist in ParE, and in many cases, are specific for GyrB. For example, these sequences are determined to be specific for GyrB in the strains shown in the following table.

| Sequence | Strain |
|---|---|
| (i) | Leclercia adecarboxylata GTC 1267 |
| | Pseudoalteromonas sp. A316 |
| | Gordonia amarae DSM 46078 |
| | Rhodococcus koreensis JCM 10743 |
| | Shigella dysenteriae GTC 786 |
| | Salmonella typhi P1 |
| | Sphingomonas sp. MBIC 5538 |
| | Serratia ficaria GTC 343 |
| | Alteromonas macleodii MBIC 1375 |
| | Vibrio fluvialis GTC 315 |
| (e) | Mycobacterium tuberculosis KPM T21 |
| | Bacteroides fragilis |
| | Acholeplasma laidlawii PG-8B |
| | Bacillus cereus JCM 2152 |
| | Treponema denticola ATCC 35405 |
| | Streptococcus pneumoniae 7785 |
| | Arthrobacter oxidans IFO 12138 |
| | Porphyromonas asaccharolytica JCM 6326 |
| | Myxococcus xanthus ER-15 |
| | Streptomyces coelicolor A3 (2) |
| (h) | Pseudomonas putida MBIC 5295 |
| | Pseudoalteromonas sp. MBIC 3307 |
| | Vibrio hollisae 89A 1962 |
| | Aeromonas hydrophila P3 |
| | Photobacterium histaminum JCM 8968 |
| | Escherichia coli W3110 |
| | Bacillus anthracis Pasteur #2 |
| | Streptococcus pneumoniae 7785 |
| | Acinetobacter calcoacelicus ATCC 23055 |
| | Acholeplasma laidlawii PG-8B |
| (k) | Pseudomonas putida MBIC 5295 |
| | Pseudoalteromonas sp. MBIC 3307 |
| | Vibrio hollisae 89A 1962 |
| | Citrobacter sp. MAM-1 |
| | Comamonas terrigena IAM 12052 |
| | Salmonella typhimurium |
| | Acinetobacter junii SEIP 14. 81 |
| | Legionella pneumophila ATCC 33152 |
| | Escherichia coli W3110 |
| | Photobacterium histaminum JCM 8968 |
| (l) | Leclercia adecarboxylata GTC 1267 |
| | Pseudoalteromonas sp. A316 |
| | Pseudomonas sp. MBIC 5390 |
| | Marinobacter sp. MBIC 4911 |
| | Shigella dysenteriae GTC 786 |
| | Salmonella typhi P1 |
| | Sphingomonas sp. MBIC 5538 |
| | Caulobacter sp. GTC 1043 |
| | Sinorhizobium fredi ATCC 35423 |
| | Comamonas sp. GTC 866 |

Accordingly, by performing PCR with the combined use of the above primers, the gyrB gene DNA only can be specifically amplified.

Two primers used for the “two steps—two fragments method” include the following amino acid sequence combinations:

| | | 2^{nd} amplification step | |
|---|---|---|---|
| | 1^{st} amplification step | fragment 1 | fragment 2 |
| Combination 1 | sequences (a) and (c) | sequences (a) and (e) | sequences (b) and (c) |
| Combination 2 | sequences (a) and (f) | sequences (a) and (e) | sequences (b) and (f) |
| Combination 3 | sequences (d) and (t) | sequences (d) and (e) | sequences (b) and (f) |

Among amino acid sequences in the above table, sequence (b) exists in GyrB of all known bacteria, and so primers synthesized based on this sequence can be applied for a wide range of microorganisms. Since sequence (b) consists of 4 amino acid residues, when PCR is performed using, as a template, DNA obtained from the microorganism with primers synthesized based on the sequence, there is a possibility to amplify DNA fragments totally irrelevant to gyrB genes. However, for the “two steps—two fragments method”, amplified gyrB gene DNA fragments are used as a template, and so the above non-specific amplification can be avoided.

Since sequence (f) is conserved in GyrB genes of many bacteria, primers synthesized based on this sequence can be applied for a wide range of microorganisms. Since sequence (f) exists also in ParE, when PCR is performed using this sequence, not only gyrB gene DNA fragments, but also parE gene DNA fragments are amplified. However, the amplification of parE gene DNA fragments can be prevented by performing PCR using primers synthesized based on sequence (e) specific for GyrB.

“Primers synthesized based on sequences (a)-(l)” mean primers encoding all or a part of amino acid sequences (a)-(l), and having a length sufficient to specifically hybridize to a specific site of a template DNA.

The nucleotide sequences of primers synthesized based on sequences (a)-(l) and amino acid sequences encoded by these nucleotide sequences are shown in the following table:

| Sequence | Amino acid sequence | Nucleotide sequence |
|---|---|---|
| (a) | SEQ ID NO: 26 | SEQ ID NO: 25 |
| | SEQ ID NO: 30 | SEQ ID NO: 29 |
| | SEQ ID NO: 54, 55, 56, 57 | SEQ ID NO: 53 |
| (b) | SEQ ID NO: 34 | SEQ ID NO: 33 |
| | SEQ ID NO: 36, 37 | SEQ ID NO: 35 |
| (c) | SEQ ID NO: 28 | SEQ ID NO: 27 |
| | SEQ ID NO: 32 | SEQ ID NO: 31 |
| | SEQ ID NO: 42 | SEQ ID NO: 41 |
| (d) | SEQ ID NO: 46, 47 | SEQ ID NO: 45 |
| (e) | SEQ ID NO: 39, 40 | SEQ ID NO: 38 |
| (f) | SEQ ID NO: 44 | SEQ ID NO: 43 |
| (g) | SEQ ID NO: 49 | SEQ ID NO: 48 |
| (h) | SEQ ID NO: 63, 64 | SEQ ID NO: 62 |
| (i) | SEQ ID NO: 59 | SEQ ID NO: 58 |
| (j) | SEQ ID NO: 66, 67, 68 | SEQ ID NO: 65 |
| (k) | SEQ ID NO: 51, 52 | SEQ ID NO: 50 |
| (l) | SEQ ID NO: 61 | SEQ ID NO: 60 |

Microorganisms applicable to the identification and detection method of the present invention include bacteria, yeast, Fungus, archaebacteria and the like.

### PREFERRED EMBODIMENTS OF THE INVENTION

### EXAMPLE 1

A PCR was performed using oligonucleotides represented by the nucleotide sequences shown in SEQ ID NOS: 25 and 27 (corresponding to the amino acid sequences of SEQ ID NOS: 26 and 28, respectively) as primers and DNA from Bacteroides vulgatus IFO 14291 strain as a template. The nucleotide sequence of the amplified DNA fragment and the amino acid sequence deduced therefrom are shown in SEQ ID NOS: 1 and 2, respectively. The PCR amplification conditions were as described below.

PCR amplification conditions:

96° C. 1 min; 48° C. 1 min; 72° C. 2 min: 3 cycles
96° C. 1 min; 48° C. 1 min; 72° C. 2 min: 3 cycles
96° C. 1 min; 48° C. 1 min; 72° C. 2 min: 30 cycles
Total: 36 cycles

| | |
|---|---|
| Primer concentration | 1 μM each |
| dATP | 200 μM each |
| Template DNA | <1 μg/100 μl |

AmpliTaq ™ and the supplied PCR Buffer (Perkin Elmer) were used.

### EXAMPLE 2

A PCR was performed using oligonucleotides represented by the nucleotide sequences shown in SEQ ID NOS: 29 and 31 (corresponding to the amino acid sequences of SEQ ID NOS: 30 and 32, respectively) as primers and DNA from Mycobacterium simiae KPM 1403 strain as a template. The PCR amplification conditions were the same as in Example 1. The nucleotide sequence of the amplified DNA fragment and the amino acid sequence deduced therefrom are shown in SEQ ID NOS: 3 and 4, respectively.

### EXAMPLE 3

A PCR was performed using oligonucleotides represented by the nucleotide sequences shown in SEQ ID NOS: 33 and 27 (corresponding to the amino acid sequences of SEQ ID NOS: 34 and 28, respectively) as primers and DNA from Chitinophaga pinensis DSM 2588 strain as a template. The PCR amplification conditions were the same as in Example 1. The nucleotide sequence of the amplified DNA fragment and the amino acid sequence deduced therefrom are shown in SEQ ID NOS: 5 and 6, respectively.

### EXAMPLE 4

A PCR was performed using oligonucleotides represented by the nucleotide sequences shown in SEQ ID NOS: 25 and 35 (corresponding to the amino acid sequences of SEQ ID NO: 26 and SEQ ID NO: 36 or 37, respectively) as primers and DNA from Flavobacterium aquatile IAM 12316 strain as a template. The PCR amplification conditions were the same as in Example 1. The nucleotide sequence of the amplified DNA fragment and the amino acid sequence deduced therefrom are shown in SEQ ID NOS: 7 and 8, respectively.

### EXAMPLE 5

A PCR was performed using oligonucleotides represented by the nucleotide sequences shown in SEQ ID NOS: 29 and 38 (corresponding to the amino acid sequences of SEQ ID NO: 30 and SEQ ID NO: 39 or 40, respectively) as primers and DNA from Mycobacterium asiaticum ATCC 25274 strain as a template. The PCR amplification conditions were the same as in Example 1. The nucleotide sequence of the amplified DNA fragment and the amino acid sequence deduced therefrom are shown in SEQ ID NOS: 9 and 10, respectively.

### EXAMPLE 6

A PCR was performed using oligonucleotides represented by the nucleotide sequences shown in SEQ ID NOS: 41 and 43 (corresponding to the amino acid sequences of SEQ ID NOS: 42 and 44, respectively) as primers and DNA from Cytophaga lytica IFO 16020 strain as a template. The PCR amplification conditions were the same as in Example 1. The nucleotide sequence of the amplified DNA fragment and the amino acid sequence deduced therefrom are shown in SEQ ID NOS: 11 and 12, respectively.

### EXAMPLE 7

A PCR was performed using oligonucleotides represented by the nucleotide sequences shown in SEQ ID NOS: 45 and 48 (corresponding to the amino acid sequences of SEQ ID NO: 46 or 47 and SEQ ID NO: 49, respectively) as primers and DNA from Synechococcus sp. PCC 6301 strain as a template. The PCR amplification conditions were the same as in Example 1. The nucleotide sequence of the amplified DNA fragment and the amino acid sequence deduced therefrom are shown in SEQ ID NOS: 13 and 14, respectively.

### EXAMPLE 8

A PCR was performed using oligonucleotides represented by the nucleotide sequences shown in SEQ ID NOS: 53 and 62 (corresponding to the amino acid sequences of SEQ ID NO: 54, 55, 56 or 57 and SEQ ID NO: 63 or 64, respectively) as primers and DNA from Caulobacter crescentus ATCC 15252 strain as a template. The PCR amplification conditions were the same as in Example 1. The nucleotide sequence of the amplified DNA fragment and the amino acid sequence deduced therefrom are shown in SEQ ID NOS: 15 and 16, respectively.

### EXAMPLE 9

A PCR was performed using oligonucleotides represented by the nucleotide sequences shown in SEQ ID NOS: 53 and 58 (corresponding to the amino acid sequences of SEQ ID NO: 54, 55, 56 or 57 and SEQ ID No: 59, respectively) as primers and DNA from Pseudomonas putida ATCC 17484 strain as a template. The PCR amplification conditions were the same as in Example 1. The nucleotide sequence of the amplified DNA fragment and the amino acid sequence deduced therefrom are shown in SEQ ID NOS: 17 and 18, respectively.

### EXAMPLE 10

A PCR was performed using oligonucleotides represented by the nucleotide sequences shown in SEQ ID NOS: 65 and 50 (corresponding to the amino acid sequences of SEQ ID NO: 66, 67 or 68 and SEQ ID NO: 51 or 52, respectively) as primers and DNA from Synechococcus sp. PCC 6301 strain as a template. The PCR amplification conditions were the same as in Example 1. The nucleotide sequence of the amplified DNA fragment and the amino acid sequence deduced therefrom are shown in SEQ ID NOS: 19 and 20, respectively.

### EXAMPLE 11

A PCR was performed using oligonucleotides represented by the nucleotide sequences shown in SEQ ID NOS: 60 and 31 (corresponding to the amino acid sequences of SEQ ID NOS: 61 and 32, respectively) as primers and DNA from Caulobacter crescentus ATCC 15252 strain as a template. The PCR amplification conditions were the same as in Example 1. The nucleotide sequence of the amplified DNA fragment and the amino acid sequence deduced therefrom are shown in SEQ ID NOS: 21 and 22, respectively.

### EXAMPLE 12

A PCR was performed using oligonucleotides represented by the nucleotide sequences shown in SEQ ID NOS: 25 and 43 (corresponding to the amino acid sequences of SEQ ID NOS: 26 and 44, respectively) as primers and DNA from an unidentified strain MBIC 1544 as a template. The PCR amplification conditions were the same as in Example 1. The nucleotide sequence of the amplified DNA fragment and the amino acid sequence deduced therefrom are shown in SEQ ID NOS: 23 and 24, respectively.

This nucleotide sequence was compared with the nucleotide sequence database possessed by the applicant. As a result, the unidentified strain MBIC 1544 was identified as Cytophaga lytica.

### EFFECT OF THE INVENTION

With the nucleotide sequence of gyr B determined by the pre sent invention, it is possible to classify or identify an unidentified microorganism strain quickly and accurately. Besides, according to the present invention, PCR primers for monitoring a specific microorganism which are needed in risk assessment in various bioprocesses can be designed easily. Also, the present invention enables highly accurate monitoring of changes in mycelial tufts.

What is claimed is:

1. A method for identifying a microorganism, comprising the following steps (1) to (5):
(1) synthesizing forward and reverse primers based on a single pair of amino acid sequences selected from the group consisting of the sequence pairs (69) and (74), (69) and (78), (72) and (71), (72) and (74), and (72) and (78);
(2) amplifying gyrB gene DNA from the microorganism using said two primers to produce a gyrB gene DNA fragment;
(3) isolating said DNA fragment;
(4) determining the nucleotide sequence of said DNA fragment; and
(5) identifying the microorganism by comparing the sequence of the amplified gyrB gene DNA fragment to known gyrB gene DNA fragment sequences.

2. The method for identifying a microorganism according to claim 1, wherein the amino acid sequence pairs that are used are sequence pairs (69) and (74), (69) and (78), (72) and (74), or (72) and (78); and said microorganism belongs to proteobacteria.

3. A method for identifying a microorganism, comprising the following steps (1) to (8):
(1) synthesizing forward and reverse primers based on a single pair of amino acid sequences (69) and (71);
(2) amplifying gyrB gene DNA from the microorganism using said two primers to produce a gyrB gene DNA fragment;
(3) synthesizing forward and reverse primers based on a single pair of amino acid sequences (69) and (73);
(4) synthesizing forward and reverse primers based on a single pair of amino acid sequences (70) and (71);
(5) amplifying the gyrB gene DNA fragment produced in step (2) using said two pairs of primers, so that two gyrB gene DNA fragments are produced;
(6) isolating said two DNA fragments;
(7) determining the nucleotide sequences of said two DNA fragments; and
(8) identifying the microorganism by comparing the sequences of said two gyrB gene DNA fragments to known gyrB gene DNA fragment sequences.

4. A method for identifying a microorganism, comprising the following steps (1) to (8):
(1) synthesizing forward and reverse primers based on a single pair of amino acid sequences (69) and (74);
(2) amplifying gyrB gene DNA from the microorganism using said two primers to produce a gyrB gene DNA fragment;
(3) synthesizing forward and reverse primers based on a single pair of amino acid sequences (69) and (73);
(4) synthesizing forward and reverse primers based on a single pair of amino acid sequences (70) and (74);
(5) amplifying the gyrB gene DNA fragment produced in step (2) using said two pairs of primers, so that two gyrB gene DNA fragments are produced;
(6) isolating said two DNA fragments;
(7) determining the nucleotide sequences of said two DNA fragments; and
(8) identifying the microorganism by comparing the sequences of said two gyrB gene DNA fragments to known gyrB gene DNA fragment sequences.

5. A method for identifying a microorganism, comprising the following steps (1) to (8):
(1) synthesizing forward and reverse primers based on a single pair of amino acid sequences (72) and (74);
(2) amplifying gyrB gene DNA from the microorganism using said two primers to produce a gyrB gene DNA fragment;
(3) synthesizing forward and reverse primers based on a single pair of amino acid sequences (72) and (73);
(4) synthesizing forward and reverse primers based on a single pair of amino acid sequences (70) and (74);
(5) amplifying the gyrB gene DNA fragment produced in step (2) using said two pairs of primers, so that two gyrB gene DNA fragments are produced;
(6) isolating said two DNA fragments;
(7) determining the nucleotide sequences of said two DNA fragments; and
(8) identifying the microorganism by comparing the sequences of said two gyrB gene DNA fragments to known gyrB gene DNA fragment sequences.

6. A method for identifying a microorganism, comprising the following steps (1) to (6):
(1) synthesizing forward and reverse primers based on a single pair of amino acid sequences (72) and (73);
(2) synthesizing forward and reverse primers based on a single pair of amino acid sequences selected from the group consisting of (76) and (71), (76) and (74), or (76) and (75);
(3) amplifying gyrB gene DNA from the microorganism using said two pairs of primers to produce two gyrB gene DNA fragments;
(4) isolating said two DNA fragments;
(5) determining the nucleotide sequences of said two DNA fragments; and
(6) identifying the microorganism by comparing the sequences of said two gyrB gene DNA fragments to known gyrB gene DNA fragment sequences.

7. A method for identifying a microorganism, comprising the following steps (1) to (6):
(1) synthesizing forward and reverse primers based on a single pair of amino acid sequences (69) and (77);
(2) synthesizing forward and reverse primers based on a single pair of amino acid sequences selected from the group consisting of (79) and (71), (79) and (74), or (79) and (75);
(3) amplifying gyrB gene DNA from the microorganism using said two pairs of primers to produce two gyrB gene DNA fragments;
(4) isolating said two DNA fragments;
(5) determining the nucleotide sequences of said two DNA fragments; and
(6) identifying the microorganism by comparing the sequences of said two gyrB gene DNA fragments to known gyrB gene DNA fragment sequences.

8. A method for identifying a microorganism, comprising the following steps (1) to (6):
(1) synthesizing forward and reverse primers based on a single pair of amino acid sequences (69) and (77);
(2) synthesizing forward and reverse primers based on a single pair of amino acid sequences selected from the group consisting of (80) and (71), (80) and (24), or (80) and (75);
(3) amplifying gyrB gene DNA from the microorganism using said two pairs of primers to produce two gyrB gene DNA fragments;
(4) isolating said two DNA fragments;
(5) determining the nucleotide sequences of said two DNA fragments; and
(6) identifying the microorganism by comparing the sequences of said two gyrB gene DNA fragments to known gyrB gene DNA fragment sequences.

9. A method for detecting a microorganism, comprising the following steps (1) to (5):
(1) synthesizing forward and reverse primers based on a single pair of amino acid sequences selected from the group consisting of the sequence pairs (69) and (74), (69) and (78), (72) and (71), (72) and (74), and (72) and (78);
(2) amplifying gyrB gene DNA from the microorganism using said two primers to produce a gyrB gene DNA fragment;
(3) isolating said DNA fragment;
(4) determining the nucleotide sequence of said DNA fragment; and
(5) detecting the microorganism by comparing the nucleotide sequence of said amplified gyrB gene DNA fragment to known gyrB gene DNA fragment sequences.

10. The method for detecting a microorganism according to claim 9, wherein the amino acid sequence pairs that are used are sequence pairs (69) and (74), (69) and (78), (72) and (74), or (72) and (78); and said microorganism belongs to proteobacteria.

11. A method for detecting a microorganism, comprising the following steps (1) to (8):
(1) synthesizing forward and reverse primers based on a single pair of amino acid sequences (69) and (71);
(2) amplifying gyrB gene DNA from the microorganism using said two primers to produce a gyrB gene DNA fragment;
(3) synthesizing forward and reverse primers based on a single pair of amino acid sequences (69) and (73);
(4) synthesizing forward and reverse primers based on a single pair of amino acid sequences (70) and (71);
(5) amplifying the gyrB gene DNA fragment produced in step (2) using said two primers to produce two gyrB gene DNA fragment;
(6) isolating said two DNA fragments;
(7) determining the nucleotide sequences of said two DNA fragments; and
(8) detecting the microorganism by comparing the sequences of said two gyrB gene DNA fragments to known gyrB gene DNA fragment sequences.

12. A method for detecting a microorganism, comprising the following steps (1) to (8):
(1) synthesizing forward and reverse primers based on a single pair of amino acid sequences (69) and (74);
(2) amplifying gyrB gene DNA from the microorganism using said two primers to produce a gyrB gene DNA fragment;
(3) synthesizing forward and reverse primers based on a single pair of amino acid sequences (69) and (73);
(4) synthesizing forward and reverse primers based on a single pair of amino acid sequences (70) and (74);
(5) amplifying the gyrB gene DNA fragment produced in step (2) using said two pairs of primers, so that two gyrB gene DNA fragments are produced;
(6) isolating said two DNA fragments;
(7) determining the nucleotide sequences of said two DNA fragments; and
(8) detecting the microorganism by comparing the sequences of said two gyrB gene DNA fragments to known gyrB gene DNA fragment sequences.

13. A method for detecting a microorganism comprising the following steps (1) to (8):
(1) synthesizing forward and reverse primers based on a single pair of amino acid sequences (72) and (74);
(2) amplifying gyrB gene DNA from the microorganism using said two primers to produce a gyrB gene DNA fragment;
(3) synthesizing forward and reverse primers based on a single pair of amino acid sequences (72) and (73);
(4) synthesizing forward and reverse primers based on a single pair of amino acid sequences (70) and (74);
(5) amplifying the gyrB gene DNA fragment produced in step (2) using said two pairs of primers, so that two gyrB gene DNA fragments are produced;
(6) isolating said two DNA fragments;
(7) determining the nucleotide sequences of said two DNA fragments; and
(8) detecting the microorganism by comparing the sequences of said two gyrB gene DNA fragments to known gyrB gene DNA fragment sequences.

14. A method for detecting a microorganism, comprising the following steps (1) to (6):
(1) synthesizing forward and reverse primers based on a single pair of amino acid sequences (72) and (73);
(2) synthesizing forward and reverse primers based on a single pair of amino acid sequences selected from the group consisting of (76) and (71), (76) and (74), or (76) and (75);
(3) amplifying gyrB gene DNA from the microorganism using said two pairs of primers to produce two gyrB gene DNA fragments;
(4) isolating said two DNA fragments;
(5) determining the nucleotide sequences of said two DNA fragments; and
(6) detecting the microorganism by comparing the sequences of said two gyrB gene DNA fragments to known gyrB gene DNA fragment sequences.

15. A method for detecting a microorganism, comprising the following steps (1) to (6):
(1) synthesizing forward and reverse primers based on a single pair of amino acid sequences (69) and (77);
(2) synthesizing forward and reverse primers based on a single pair of amino acid sequences selected from the group consisting of (79) and (71), (79) and (74), or (79) and (75);
(3) amplifying gyrB gene DNA from the microorganism using said two pairs of primers to produce two gyrB gene DNA fragments;
(4) isolating said two DNA fragments;
(5) determining the nucleotide sequences of said two DNA fragments; and
(6) detecting the microorganism by comparing the sequences of said two gyrB gene DNA fragments to known gyrB gene DNA fragment sequences.

16. A method for detecting a microorganism, comprising the following steps (1) to (6):
(1) synthesizing forward and reverse primers based on a single pair of amino acid sequences (69) and (77);
(2) synthesizing forward and reverse primers based on a single pair of amino acid sequences selected from the group consisting of (80) and (71), (80) and (74), or (80) and (75);
(3) amplifying gyrB gene DNA from the microorganism using said two pairs of primers to produce two gyrB gene DNA fragments;
(4) isolating said two DNA fragments;
(5) determining the nucleotide sequences of said two DNA fragments; and
(6) detecting the microorganism by comparing the sequences of said two gyrB gene DNA fragments to known gyrB gene DNA fragment sequences.


SEQUENCE LISTING (82 sequences)

SEQ ID NO: 1 - DNA, 1212 bp, Bacteroides vulgatus
  CDS (1)...(1212)
gacaaaggttcttacaaggtttcaggcggtctgcacggtgtaggtgtttcttgtgtgaacgccttgtctactcacatgaccacacaggtattccgcggtggcaagatctaccagcaggaatacagctgcggacatcctttgtattctgtaaaagaagtaggaacagctgatattaccggaacaaaacagactttctggccggatgataccatcttcactgttaccgaatataagtttgacattctacaggcacgtatgcgtgaattggcctacttgaacaaaggtatcaccatttcactgaccgaccgccggatcaaagaagaagatggcagcttcaagaaagaaatattccattcggacgaaggagtgaaagagtttgtacgtttcctgaaccgtaacaacgaagcgctgattaatgatgtcatttatctgaataccgaaaaaaacaatacccccattgaatgtgccatcatgtacaatacaggctatcgtgaaagcctgcattcgtatgtaaacaatatcaatacaatagaaggcggtacacacgaggccggtttccgcagcgcattaacccgtgtactgaagaaatatgcggaagataccaaagcactggaaaaagcaaaagtcgagatttcgggagaggacttccgcgaaggcttgattgccgtcatttcagtgaaagtagccgagccgcagttcgaaggacagaccaagaccaagctgggcaacagcgaagtgagtggtgccgtgaaccaagctgtaggcgaagcgcttacatattatctggaagaacatccgaaagaagcaaaacagattgttgacaaagtgatcctggctgcaacagcgcgtatcgccgcacgcaaggcacgtgaatctgttcaaagaaagagtccgatgggcggtggcggactgccgggcaaactggccgactgctcgagccgtaatccggaggaatgtgaactattcctggtcgagggtgactcggcaggtggttctgccaagcaaggacgtagccgtgccttccaggcaattctacctttgaggggtaaaatcctgaatgtggaaaaagcgatgtggcacaaggcttttgaaagcgatgaggtcaataatatcatcaccgccctgggtgtccgtttcggtgtggacggaaatgatgacagcaaaaaagcgaacatcgacaagctgcgttatcacaaagtggtgatcatgacc

SEQ ID NO: 2 - protein, 404 aa, Bacteroides vulgatus
DKGSYKVSGGLHGVGVSCVNALSTHMTTQVFRGGKIYQQEYSCGHPLYSVKEVGTADITGTKQTFWPDDTIFTVTEYKFDILQARMRELAYLNKGITISLTDRRIKEEDGSFKKEIFHSDEGVKEFVRFLNRNNEALINDVIYLNTEKNNTPIECAIMYNTGYRESLHSYVNNINTIEGGTHEAGFRSALTRVLKKYAEDTKALEKAKVEISGEDFREGLIAVISVKVAEPQFEGQTKTKLGNSEVSGAVNQAVGEALTYYLEEHPKEAKQIVDKVILAATARIAARKARESVQRKSPMGGGGLPGKLADCSSRNPEECELFLVEGDSAGGSAKQGRSRAFQAILPLRGKILNVEKAMWHKAFESDEVNNIITALGVRFGVDGNDDSKKANIDKLRYHKVVIMT

SEQ ID NO: 3 - DNA, 1263 bp, Mycobacterium simiae
  CDS (1)...(1263)
ggggagaacagtggctacaccgtcagcggcgggttgcacggggtcggagtgtcggtggtcaacgccctgtccacccgcctggaagtcaacgtcaagcgtgacggctatgagtggttccagtactacgaccgggcggtgcccggcaccctcaagcaaggcgaggcgaccaagaagaccggcaccacgatccggttctgggccgatcctgagatcttcgaaaccacccagtacgacttcgagacggtggcgcgccggttgcaggaaatggcgttcctcaacaagggcctgaccatcaacctcaccgacgaacgtgtcgagcaggacgaggtggtcgatgaggtggttagcgacaccgccgaggcgccgaagtcagccgaggagcaggcggccgaatcggccaagccgcacaaggtcaagcaccgcacgttccactacccgggtgggttggtggatttcgtcaagcacatcaatcgcaccaaaaacccgatccagcagagcgtcatcgacttcgacggcaaaggaaccgggcacgaagtcgagatcgcgatgcagtggaacggtggttattcggagtcggtgcacaccttcgccaacaccatcaacacccatgagggcggcacccacgaggagggcttccgcagcgcgctgacctcggtggtgaacaagtacgccaaagacaagaagctgctcaaggacaaggatcccaacctcaccggcgacgacatccgagaagggctggccgcggtgatctccgtgaaggtcgccgagccgcagttcgagggccagactaagacgaaactcggcaacaccgaggtcaagtcgtttgtccagaaagtctgtaacgaacaactcactcactggttcgaggcgaacccgtcggaagctaaaaccgttgtaaacaaggcggtttcgtcggcccaggcccgcattgcggcgcgtaaggcgcgggagttggtgcggcgtaagagtgctacggatttgggtgggttgccgggcaagttggctgattgccgctcgacggatccgcggaagtctgagctgtatgtggtggaaggtgattccgcgggtgggtcggcgaaaagtgggcgtgattcgatgttccaggcgatcttgccgctgcgcggcaagatcatcaacgtcgaaaaggcccgcatcgatcgggtgctgaaaaacaccgaagtccaggccatcatcaccgcgctgggcaccggcatccacgacgaattcgacatcaccaaactgcgttaccacaagatcgtgttg

SEQ ID NO: 4 - protein, 421 aa, Mycobacterium simiae
GENSGYTVSGGLHGVGVSVVNALSTRLEVNVKRDGYEWFQYYDRAVPGTLKQGEATKKTGTTIRFWADPEIFETTQYDFETVARRLQEMAFLNKGLTINLTDERVEQDEVVDEVVSDTAEAPKSAEEQAAESAKPHKVKHRTFHYPGGLVDFVKHINRTKNPIQQSVIDFDGKGTGHEVEIAMQWNGGYSESVHTFANTINTHEGGTHEEGFRSALTSVVNKYAKDKKLLKDKDPNLTGDDIREGLAAVISVKVAEPQFEGQTKTKLGNTEVKSFVQKVCNEQLTHWFEANPSEAKTVVNKAVSSAQARIAARKARELVRRKSATDLGGLPGKLADCRSTDPRKSELYVVEGDSAGGSAKSGRDSMFQAILPLRGKIINVEKARIDRVLKNTEVQAIITALGTGIHDEFDITKLRYHKIVL

SEQ ID NO: 5 - DNA, 660 bp, Chitinophaga pinensis
  CDS (1)...(660)
gtagcaggcttccgccgtgcgataacccgtatcttcaagagctatggtgataagaacaaaatgttcgaaaaaaccaagatcgaagtaacaggtgatgacttccgtgaaggtctgagcgctatcatcagcgtaaaagtacctgaaccacagttcgaaggccagaccaaaaccaaactcggtaactccgatgtaatgggggttgtggacagttccgtagcagccgtactggatgcctacctggaagaacatccccgcgaagccaagatcattatcaataaagtggtactggcagcacaggcgcgtgaagcagcccgtaaagcacgccagatggtacagcgtaagagcgtactgagtggaagcggcttgcctggtaaactggctgactgctctgaaaatgatcctgaaaaatgtgaactgtacctggtagagggtgactccgcaggtggtacggctaaacaaggacgtaaccgtagcttccaggcgatcctgccgctcaggggtaaaatcctgaacgtggagaaagccatggagcataagatatatgagaatgaggagattaaaaacatcttcaccgcacttggtgtaaccatcggtacggaagaagatgacaaagccctcaacctctccaaactgcgctatcacaaactgatcatcatgacg

SEQ ID NO: 6 - protein, 220 aa, Chitinophaga pinensis
VAGFRRAITRIFKSYGDKNKMFEKTKIEVTGDDFREGLSAIISVKVPEPQFEGQTKTKLGNSDVMGVVDSSVAAVLDAYLEEHPREAKIIINKVVLAAQAREAARKARQMVQRKSVLSGSGLPGKLADCSENDPEKCELYLVEGDSAGGTAKQGRNRSFQAILPLRGKILNVEKAMEHKIYENEEIKNIFTALGVTIGTEEDDKALNLSKLRYHKLIIMT

SEQ ID NO: 7 - DNA, 537 bp, Flavobacterium aquatile
  CDS (1)...(537)
gataaagattcttataaagtttcgggtggacttcacggagttggtgtttcttgcgttaatgcactttctgataacctaaaagcaaccgtttttagagacggaaaagtgtacgagcaagaatatgaaaaaggtaaagcaatgtatccggttaagcaagttggtgaaacaacaaagcgaggaacaatggttacttttcatcctgataaaaccatttttactcaaacaattgagtattcttatgatacacttgcagcacgtatgcgtgaattatctttcctgaataaaggaattacaatcacacttacagataaaagacatactaaagacaacggcgattttgaaggtgaagtttttcattctaaagaagggcttaaagaattcgttcgatttttagatgctggtagagaaccaattatttctcacgtaataagcatggagcacgaaaaaggagaagttcctgttgaggttgctcttgtttacaatacaagttactccgaaaatattttctcttacgtaaataatattaacacgcacgaa

SEQ ID NO: 8 - protein, 179 aa, Flavobacterium aquatile
DKDSYKVSGGLHGVGVSCVNALSDNLKATVFRDGKVYEQEYEKGKAMYPVKQVGETTKRGTMVTFHPDKTIFTQTIEYSYDTLAARMRELSFLNKGITITLTDKRHTKDNGDFEGEVFHSKEGLKEFVRFLDAGREPIISHVISMEHEKGEVPVEVALVYNTSYSENIFSYVNNINTHE

SEQ ID NO: 9 - DNA, 783 bp, Mycobacterium asiaticum
  CDS (1)...(783)
ggcgagaacagcggctacaccgtcagcggtgggttgcacggagtgggcgtgtcggtggtcaacgcgctgtccacccgcctggaggtcaccatcaagcgcgacgggcacgagtggtttcagtactacgaccgcgccgtgcccggaaccctcaagcagggcgaggccaccaagaagaccggaaccacgatcaggttctgggcggaccccgaaatcttcgaaaccacacagtacgacttcgagaccgtggcgcggcggctgcaggagatggccttcctcaacaagggcctcaccatcaacctcaccgacgaacgagtggagcaggacgaggtcgtcgacgaggtcgtcagcgacaccgccgaggcaccgaagtccgccgaagagaaggccgcggaatcgactgcgccacacaaggtcaagcaccgcaccttccactaccccggcggtctggtcgacttcgtcaagcacatcaaccgcaccaagagcccgatccagcagagcgtcatcgatttcgacggcaagggcaccggccacgaggtcgagatcgccatgcagtggaacggcggctactcggagtccgtccacaccttcgccaacaccatcaacacgcacgagggcggcacccacgaggagggcttccgcagcgcgctgacgtcggtggtgaacaagtacgccaaagacaagaaactgctgaaggacaaagatcccaacctcaccggtgacgacatccgtgagggcttggccgcggtcatctcggtgaaggtcgccgagccacagttcgaaggc

SEQ ID NO: 10 - protein, 261 aa, Mycobacterium asiaticum
GENSGYTVSGGLHGVGVSVVNALSTRLEVTIKRDGHEWFQYYDRAVPGTLKQGEATKKTGTTIRFWADPEIFETTQYDFETVARRLQEMAFLNKGLTINLTDERVEQDEVVDEVVSDTAEAPKSAEEKAAESTAPHKVKHRTFHYPGGLVDFVKHINRTKSPIQQSVIDFDGKGTGHEVEIAMQWNGGYSESVHTFANTINTHEGGTHEEGFRSALTSVVNKYAKDKKLLKDKDPNLTGDDIREGLAAVISVKVAEPQFEG

SEQ ID NO: 11 - DNA, 195 bp, Cytophaga lytica
  CDS (1)...(195)
agccacattgaaactttaattcttacattcttcttccgttttatgcgagaactaatagaaggcggacacgtttacatagcaacaccacctttatatttagttaaaaaaggaactaaaaagcgttatgcttggaatgataaagaacgagatgaaatagcagatagctttaatggtagtgtaggtatccaaagatat

SEQ ID NO: 12 - protein, 65 aa, Cytophaga lytica
SHIETLILTFFFRFMRELIEGGHVYIATPPLYLVKKGTKKRYAWNDKERDEIADSFNGSVGIQRY

SEQ ID NO: 13 - DNA, 1170 bp, Synechococcus sp.
  CDS (1)...(1170)
gtggtggacaacgccgtcgacaaagccttggcgggctactgcaataccattgatgttcgtctgctcaaagacggctcctgccaagtcaccgataacggtcgcggcattcccacagatattcacccccaaaccgggaagtctgctctcgaaaccgtgctgacgattctgcacgcgggcggcaagtttggcggtggcggttataaggtgtcggggggtctgcacggcgtcggtgtgtctgtcgtcaacgccctctcagaatatgtcgaagtcaccgtgtggcgggaaggcaaaacccaccaacagcgctttgaacagggcaacccgatcggggagttgcaagttgccccggatgccgacgatcgccgcgggacacaagttcgtttcaaaccagacgccacgatcttttctgaaacaaccgagttcgattacggcaccctagcaagccgattgaaggagctagcctatctgaatgcgggcgtccgcatcgactttaccgatgagcggctgcagctcaccaagaatcacgagccccatcaagaaacctattactttgaaggcggtattcgcgaatacgtcgcctacatgaataccgataaacaggcgctgcactcagagattatctttgtgcaatccgaaaaagatggcgtccaagttgaagctgcattgcaatggtgcgttgacgcctacagcgacaacattctgggctttgccaacaacatccgcacgattgacggcggcacccatattgaggggctcaaaactgttctgacgcggacgatgaacacgatcgcccgcaaacggaataaacgcaaggatgccgacaataacctgtcgggcgagaatattcgcgaagggttaacagcgatcgtttcggtcaaagttccggatccggaatttgaagggcaaaccaaaacaaagctcggcaataccgaagttcgcggcatcgtcgatacgctcgtgggcgaaacgttgacggaatatctggaattccatcccagcgttgccgatttgatcctcgaaaaagcgattcaagcctttaatgcggctgaggcagcgcgacgggcacgggaattggtgcgtcgcaaatcagtgctggaatcttcgacattgcccggtaaattagcagactgttccagtcgcgatcccggtgaatctgaaatcttcatcgtg

SEQ ID NO: 14 - protein, 390 aa, Synechococcus sp.
VVDNAVDKALAGYCNTIDVRLLKDGSCQVTDNGRGIPTDIHPQTGKSALETVLTILHAGGKFGGGGYKVSGGLHGVGVSVVNALSEYVEVTVWREGKTHQQRFEQGNPIGELQVAPDADDRRGTQVRFKPDATIFSETTEFDYGTLASRLKELAYLNAGVRIDFTDERLQLTKNHEPHQETYYFEGGIREYVAYMNTDKQALHSEIIFVQSEKDGVQVEAALQWCVDAYSDNILGFANNIRTIDGGTHIEGLKTVLTRTMNTIARKRNKRKDADNNLSGENIREGLTAIVSVKVPDPEFEGQTKTKLGNTEVRGIVDTLVGETLTEYLEFHPSVADLILEKAIQAFNAAEAARRARELVRRKSVLESSTLPGKLADCSSRDPGESEIFIV

SEQ ID NO: 15 - DNA, 696 bp, Caulobacter crescentus
  CDS (1)...(696)
cagaacagctacaaggtctcgggcggtctgcacggcgtgggcgtctcggtcgtgaacgccctgtcggattggctggagctgctgatccaccgcaacggcaaggtccaccagatgcgcttcgagcgcggcgacgcggtcacctcgctgaaggtcaccggcgactcgcccgtgcggaccgagggccccaaggccggcgagaccctgaccggtacggaagttacgttctttccgtcgaaggacaccttcgccttcatcgaattcgaccggaagacgctggagcaccgcctgcgcgagctggccttcctgaactcgggcgtgacgatctggttcaaggaccatcgcgacgtcgagccgtgggaagagaagctgttctacgagggcggcatcgaggccttcgtgcgccacctcgacaaggccaagacgccgctgctgaaggccccgatcgccgtcaagggcgtcaaggacaaggtcgagatcgacctggccctgtggtggaacgacagctaccacgagcagatgctgtgcttcaccaacaacatcccgcagcgggatggcggcacgcacctgtcggcctttcgcgcggccctgacccggatcatcaccagctacgccgagagctccggcatcctgaagaaggaaaaggtcagcctgggcggcgaagacagccgcgagggcctgacctgcgtgctgtcggtc

SEQ ID NO: 16 - protein, 232 aa, Caulobacter crescentus
QNSYKVSGGLHGVGVSVVNALSDWLELLIHRNGKVHQMRFERGDAVTSLKVTGDSPVRTEGPKAGETLTGTEVTFFPSKDTFAFIEFDRKTLEHRLRELAFLNSGVTIWFKDHRDVEPWEEKLFYEGGIEAFVRHLDKAKTPLLKAPIAVKGVKDKVEIDLALWWNDSYHEQMLCFTNNIPQRDGGTHLSAFRAALTRIITSYAESSGILKKEKVSLGGEDSREGLTCVLSV

SEQ ID NO: 17 - DNA, 888 bp, Pseudomonas putida
  CDS (1)...(888)
ggcggcctgcacggtgtaggcgtgtcggtagtgaacgcactgtctgaagagctcgtcctcaccgttcgccgtagcggcaagatctgggaacagacctacgtccatggtgttccgcaggaaccgatgaagatcgttggcgacagcgaaaccaccggcacccagatccacttcaaggcttccagcgaaaccttcaagaacatccacttcagctgggacatcctggccaagcggattcgtgaactgtccttcctcaactccggtgtcggcatcgtcctcaaggatgagcgcagcggcaaggaagaactgttcaagtacgaaggcggcttgcgcgcgttcgttgaatacctgaacaccaacaagaccccggtcaaccaggtgttccatttcaacatccagcgcgaagacggcatcggcgtagaaatcgccctgcagtggaacgacagcttcaacgagaacctgttgtgcttcaccaacaacattccgcagcgcgatggcggtactcacctggtgggtttccgttccgccctgacgcgtaacctcaatacgtatatcgaagccgaaggcctggcgaagaagcacaaggtcgcgaccaccggtgacgatgcccgtgaaggcctggccgcgatcatttcggtaaaagtgccggatccgaagttcagctcccagaccaaggacaagctggtttcttccgaagtgaagaccgcggtcgaacaggaaatgggcaagtacttctccgacttcctgctggaaaacccgaacgaagccaagctggttgtcggcaagatgatcgacgcggcacgtgctcgtgaagcggcgcgcaagacccgtgagatgacccgccgcaaaggcgcgctggacatcgccggcctgccgggcaaactggctgac

SEQ ID NO: 18 - protein, 296 aa, Pseudomonas putida
GGLHGVGVSVVNALSEELVLTVRRSGKIWEQTYVHGVPQEPMKIVGDSETTGTQIHFKASSETFKNIHFSWDILAKRIRELSFLNSGVGIVLKDERSGKEELFKYEGGLRAFVEYLNTNKTPVNQVFHFNIQREDGIGVEIALQWNDSFNENLLCFTNNIPQRDGGTHLVGFRSALTRNLNTYIEAEGLAKKHKVATTGDDAREGLAAIISVKVPDPKFSSQTKDKLVSSEVKTAVEQEMGKYFSDFLLENPNEAKLVVGKMIDAARAREAARKTREMTRRKGALDIAGLPGKLAD

SEQ ID NO: 19 - DNA, 531 bp, Synechococcus sp.
  CDS (1)...(531)
ttggtgcgtcgcaaatcagtgctggaatcttcgacattgcccggtaaattagcagactgttccagtcgcgatcccggtgaatctgaaatcttcatcgtggaaggggattcggcaggtggcagtgctaaacaggggcgcgatcgccgcttccaagccatcctgcctctgcgcggcaaaatcctcaacatcgagaaaacggacgatgccaaaatctacaaaaacactgagatccaagccctgattacagcgctgggcctcggaattaaaggggaggaatttgatgcttcccaactgcgctaccaccgtattgtgatcatgactgacgcggacgtcgatggtgcgcacatccgtaccctcttgctcaccttcttctatcgctatcagcgatcgctgctggagcagggctacatgtacattgcctgcccgccgctgtacaagttggagcggggacgtaatcactactattgctacaacgaacgcgaactgcaggaacggattgcgacgttccctgaaaacgccaactatacgatt

SEQ ID NO: 20 - protein, 177 aa, Synechococcus sp.
LVRRKSVLESSTLPGKLADCSSRDPGESEIFIVEGDSAGGSAKQGRDRRFQAILPLRGKILNIEKTDDAKIYKNTEIQALITALGLGIKGEEFDASQLRYHRIVIMTDADVDGAHIRTLLLTFFYRYQRSLLEQGYMYIACPPLYKLERGRNHYYCYNERELQERIATFPENANYTI

SEQ ID NO: 21 - DNA, 660 bp, Caulobacter crescentus
  CDS (1)...(660)
cgggatggcggcacgcacctgtcggcctttcgcgcggccctgacccggatcatcaccagctacgccgagagctccggcatcctgaagaaggaaaaggtcagcctgggcggcgaagacagccgcgagggcctgacctgcgtgctgtcggtcaaggtcccggatccgaagttcagctcgcagaccaaggacaagctggtctcgtccgaagtgcgccccgccgttgagggcctggtgtcggaaggtctctcgacctggttcgaggaacatccgaacgaggccaaggcgatcgtgaccaagatcgccgaggccgccgccgcccgcgaggccgcccgcaaggcgcgagagctgacccgccgcaagagcgcgctcgacatcaccagcctgcccggcaagctcgccgactgctcggaacgcgatccggccaagtccgagatcttcatcgtcgagggcgactcggcgggcggctcggccaagcaggcccgcaaccgcgacaaccaggccgttctgcccctgcgcggcaagatcctgaacgtcgagcgggcccgcttcgacaagatgctgtcgtccgaccagatcggcacgctgatcaccgccctgggcgcggggatcggccgcgacgacttcaacccggacaaggtgcgctaccacaagatcgtgctg

SEQ ID NO: 22 - protein, 220 aa, Caulobacter crescentus
RDGGTHLSAFRAALTRIITSYAESSGILKKEKVSLGGEDSREGLTCVLSVKVPDPKFSSQTKDKLVSSEVRPAVEGLVSEGLSTWFEEHPNEAKAIVTKIAEAAAAREAARKARELTRRKSALDITSLPGKLADCSERDPAKSEIFIVEGDSAGGSAKQARNRDNQAVLPLRGKILNVERARFDKMLSSDQIGTLITALGAGIGRDDFNPDKVRYHKIVL

SEQ ID NO: 23 - DNA, 1422 bp, Cytophaga lytica
  CDS (1)...(1422)
gataaagattcatacaaagtatctggtggtttacacggtgtaggtgtatcttgtgtaaacgcattatctaataatttaaaagctactgtttacagagaaggtaaaatatgggagcaagagtatgaaagaggtaaggctttatatccggtaaaaagtattggagaaacagaggaaacaggtactatagttactttttacccagatgatactatatttacacaaactacagagtataattatgaaacgctttctaacagaatgcgagagttggcttaccttaataagggagttacaattagcattacagataagagagttaaagatgaaaagggagagtttttatctgaagttttttactctgaagaaggactaaaagaatttattaagtttttagacggtaacagagaacaactaatacgtgatgttgtttcaatggaaggtgaaaaaaacggaattcctgttgaggttgcaatggtgtacaatacatcatattcagaaaatcttcactcttacgtaaataatattaatacacatgaaggtggtacacacctttcaggttttagaagaggtttaacatcaaccttaaaaaagtatgcagatgcatctggaatgttagacaaattaaagtttgagattcagggagatgattttagagaaggtttaacggctattgtgtctgttaaagttgcagaacctcagtttgaagggcaaacaaaaactaaattaggtaacagagaagtttcttctgcagtgagccaagctgtatcagaaatgcttaccaactatttagaagaaaacccagatgatgctaaggtaattgtacaaaaagtcattttggcagcgcaagcacgtcatgcggctacaaaagcccgtgaaatggtacagcgtaaaacggtaatgagtataggtggtttaccagggaaattatcagactgttctgagcaagatgctacaaaatgcgaagtattccttgtagagggagattcggcgggtggtactgctaaacaaggtagggacagaaactttcaggcaatattaccgcttcgtggtaaaatcttaaatgttgaaaaagcaatgcaacataaggtttttgaaaacgaagaaataaaaaatatttacacagctttaggtgttactattggtacagaagaagatagtaaagccttaaacttagaaaaattaagataccataaagtagttattatgtgtgatgccgatgtagatggtagccacattgaaactttaatccttacattcttcttccgttttatgagggagttaatagaaggcggtcacgtttatatagcaaccccacctttatacttggtaaaaaagggaacaaaaaaacgttatgcttggaatgataaagaacgagatgagatagcagaaagctttaatggtagtgttggtatacaaagatat

SEQ ID NO: 24 - protein, 474 aa, Cytophaga lytica
DKDSYKVSGGLHGVGVSCVNALSNNLKATVYREGKIWEQEYERGKALYPVKSIGETEETGTIVTFYPDDTIFTQTTEYNYETLSNRMRELAYLNKGVTISITDKRVKDEKGEFLSEVFYSEEGLKEFIKFLDGNREQLIRDVVSMEGEKNGIPVEVAMVYNTSYSENLHSYVNNINTHEGGTHLSGFRRGLTSTLKKYADASGMLDKLKFEIQGDDFREGLTAIVSVKVAEPQFEGQTKTKLGNREVSSAVSQAVSEMLTNYLEENPDDAKVIVQKVILAAQARHAATKAREMVQRKTVMSIGGLPGKLSDCSEQDATKCEVFLVEGDSAGGTAKQGRDRNFQAILPLRGKILNVEKAMQHKVFENEEIKNIYTALGVTIGTEEDSKALNLEKLRYHKVVIMCDADVDGSHIETLILTFFFRFMRELIEGGHVYIATPPLYLVKKGTKKRYAWNDKERDEIAESFNGSVGIQRY

SEQ ID NO: 25 - DNA, 38 bp, Artificial Sequence
  oligonucleotide for PCR
tgtaaaacgacggccagtcaygcnggnggnaarttyga

SEQ ID NO: 26 - protein, 7 aa, Artificial Sequence
  synthetically generated peptide
HAGGKFD

SEQ ID NO: 27 - DNA, 36 bp, Artificial Sequence
  oligonucleotide for PCR
ctgcgttcgtatatgagcnccrtcnacrtcngcrtc

SEQ ID NO: 28 - protein, 12 aa, Artificial Sequence
  synthetically generated peptide
DADVDGAHIRTL

SEQ ID NO: 29 - DNA, 41 bp, Artificial Sequence
  oligonucleotide for PCR
gaagtcatcatgaccgttctgcaygsnggnggnaarttygg

SEQ ID NO: 30 - protein, 14 aa, Artificial Sequence
  synthetically generated peptide
EVLMTVLHAGGKFG

SEQ ID NO: 31 - DNA, 44 bp, Artificial Sequence
  oligonucleotide for PCR
agcagggtacggatgtgcgagccrtcnacrtcngcrtcngtgat

SEQ ID NO: 32 - protein, 15 aa, Artificial Sequence
  synthetically generated peptide
MTDADVDGSHIRTLL

SEQ ID NO: 33 - DNA, 32 bp, Artificial Sequence
  oligonucleotide for PCR
caggaaacagctatgaccarrtgngtnccncc

SEQ ID NO: 34 - protein, 5 aa, Artificial Sequence
  synthetically generated peptide
GGTHL

SEQ ID NO: 35 - DNA, 34 bp, Artificial Sequence
  oligonucleotide for PCR
gcaacgagatcaacactcmngarggnggnacnca

SEQ ID NO: 36 - protein, 11 aa, Artificial Sequence
  synthetically generated peptide
NNINTHEGGTH

SEQ ID NO: 37 - protein, 11 aa, Artificial Sequence
  synthetically generated peptide
NNINTPEGGTH

SEQ ID NO: 38 - DNA, 35 bp, Artificial Sequence
  oligonucleotide for PCR
tgtaaaacgacggccagtaryttnkyyttngtytg

SEQ ID NO: 39 - protein, 6 aa, Artificial Sequence
  synthetically generated peptide
QTKTKL

SEQ ID NO: 40 - protein, 6 aa, Artificial Sequence
  synthetically generated peptide
QTKDKL

SEQ ID NO: 41 - DNA, 35 bp, Artificial Sequence
  oligonucleotide for PCR
taggctagctgaccgtaagaygcngayrtngaygg

SEQ ID NO: 42 - protein, 6 aa, Artificial Sequence
  synthetically generated peptide
DADVDG

SEQ ID NO: 43 - DNA, 36 bp, Artificial Sequence
  oligonucleotide for PCR
ccatagctgcgtagcattcatytcnccnarnccytt

SEQ ID NO: 44 - protein, 12 aa, Artificial Sequence
  synthetically generated peptide
KGLGEMNATQLW

SEQ ID NO: 45 - DNA, 41 bp, Artificial Sequence
  oligonucleotide for PCR
caggaaacagctatgaccaarmgnccngsnatgtayathgg

SEQ ID NO: 46 - protein, 8 aa, Artificial Sequence
  synthetically generated peptide
KRPAMYIG

SEQ ID NO: 47 - protein, 8 aa, Artificial Sequence
  synthetically generated peptide
KRPGMYIG

SEQ ID NO: 48 - DNA, 38 bp, Artificial Sequence
  oligonucleotide for PCR
tgtaaaacgacggccagtccnccngcnswrtcnccytc

SEQ ID NO: 49 - protein, 7 aa, Artificial Sequence
  synthetically generated peptide
EGDSAGG

SEQ ID NO: 50 - DNA, 39 bp, Artificial Sequence
  oligonucleotide for PCR
tgtaaaacgacggccagtcatngtngtntcccanarytg

SEQ ID NO: 51 - protein, 7 aa, Artificial Sequence
  synthetically generated peptide
QLWETTM

SEQ ID NO: 52 - protein, 7 aa, Artificial Sequence
  synthetically generated peptide
QLWDTTM

SEQ ID NO: 53 - DNA, 41 bp, Artificial Sequence
  oligonucleotide for PCR
gaagtcatcatgaccgttctgcaygcnggnggnaarttyga

SEQ ID NO: 54 - protein, 14 aa, Artificial Sequence
  synthetically generated peptide
EVIMTVLHAGGKFD

SEQ ID NO: 55 - protein, 14 aa, Artificial Sequence
  synthetically generated peptide
EVIMTVLHAGGKFN

SEQ ID NO: 56 - protein, 14 aa, Artificial Sequence
  synthetically generated peptide
EVIMTVLHAGGKFE

SEQ ID NO: 57 - protein, 14 aa, Artificial Sequence
  synthetically generated peptide
EVIMTVLHAGGKFK

SEQ ID NO: 58 - DNA, 38 bp, Artificial Sequence
  oligonucleotide for PCR
tgtaaaacgacggccagtgcnggrtcyttytcytgrca

SEQ ID NO: 59 - protein, 7 aa, Artificial Sequence
  synthetically generated peptide
CQEKDPA

SEQ ID NO: 60 - DNA, 40 bp, Artificial Sequence
  oligonucleotide for PCR
gaagtcatcatgaccgttctgcaacnaayaayathccnca

SEQ ID NO: 61 - protein, 6 aa, Artificial Sequence
  synthetically generated peptide
TNNIPQ

SEQ ID NO: 62 - DNA, 38 bp, Artificial Sequence
  oligonucleotide for PCR
tgtaaaacgacggccagtaayttnggntcnggnacytt

SEQ ID NO: 63 - protein, 7 aa, Artificial Sequence
  synthetically generated peptide
KVPDPKF

SEQ ID NO: 64 - protein, 7 aa, Artificial Sequence
  synthetically generated peptide
KVPEPKF

SEQ ID NO: 65 - DNA, 35 bp, Artificial Sequence
  oligonucleotide for PCR
caggaaacagctatgaccgcnmrnmrngcnmgnga

SEQ ID NO: 66 - protein, 6 aa, Artificial Sequence
  synthetically generated peptide
ARRARE

SEQ ID NO: 67 - protein, 6 aa, Artificial Sequence
  synthetically generated peptide
ARKARE

SEQ ID NO: 68 - protein, 6 aa, Artificial Sequence
  synthetically generated peptide
AKKARE

SEQ ID NO: 69 - protein, 19 aa, Artificial Sequence
  synthetically generated peptide
XXXXXXXTXLHAGGKFXXX

SEQ ID NO: 70 - protein, 4 aa, Artificial Sequence
  synthetically generated peptide
GGTH

SEQ ID NO: 71 - protein, 15 aa, Artificial Sequence
  synthetically generated peptide
XMTDADVDGXHIRTL

SEQ ID NO: 72 - protein, 11 aa, Artificial Sequence
  synthetically generated peptide
RKRPXMYIGXT

SEQ ID NO: 73 - protein, 6 aa, Artificial Sequence
  synthetically generated peptide
QXXXKL

SEQ ID NO: 74 - protein, 9 aa, Artificial Sequence
  synthetically generated peptide
XKGLGEMNX

SEQ ID NO: 75 - protein, 9 aa, Artificial Sequence
  synthetically generated peptide
VEGDSAGGS

SEQ ID NO: 76 - protein, 7 aa, Artificial Sequence
  synthetically generated peptide
KXPDPXF

SEQ ID NO: 77 - protein, 14 aa, Artificial Sequence
  synthetically generated peptide
LPGKLADCXXXDPX

SEQ ID NO: 78 - protein, 9 aa, Artificial Sequence
  synthetically generated peptide
QLXXTTXXP

SEQ ID NO: 79 - protein, 6 aa, Artificial Sequence
  synthetically generated peptide
AXXARE

SEQ ID NO: 80 - protein, 7 aa, Artificial Sequence
  synthetically generated peptide
FTNNIXX

SEQ ID NO: 81 - protein, 7 aa, Pseudomonas putida
HAGGKFD

SEQ ID NO: 82 - protein, 8 aa, Pseudomonas putida
MTDADVDG